(19) United States
(12) Patent Application Publication
(10) Pub. No.: US 2006/0211972 A1
(43) Pub. Date: Sep. 21, 2006

(54) Wound dressing

(76) Inventors: Brian Nielsen, Goerloese (DK); Maria Severin, Fredensborg (DK)

Correspondence Address: JACOBSON HOLMAN PLLC, 400 SEVENTH STREET N.W., SUITE 600, WASHINGTON, DC, 20004, US

(21) Appl. No.: 10/550,734

(22) PCT Filed: Mar. 24, 2004
(86) PCT No.: PCT/DK04/00204

§ 371 Date: Sep. 26, 2005

(30) Foreign Application Priority Data
Mar. 28, 2003 (DK) PA 2003 00477
Jul. 11, 2003 (DK) PA 2003 01063

(51) Int. Cl. A61F 13/00

(52) U.S. Cl. 602/48

(57) ABSTRACT

A wound dressing comprising a web of gel-forming fibers or fibers soluble in wound exudates, attached to a reinforcing layer wherein the density of the web is in the range of 5-60 g/m². The dressing may serve as a wound-contacting layer, providing moist wound healing combined with easy, non-traumatic removal from the wound site. The dressing may further comprise one or more active ingredients.

This is a nationalization of PCT/DK04/000204 filed Mar. 24, 2004 and published in English.

### BACKGROUND OF THE INVENTION

1. Field of the Invention

The invention relates to a wound dressing, especially a wound dressing that provides moist wound healing.

In the treatment of wounds, especially chronic wounds such as pressure sores, leg ulcers and diabetic wounds, it is important to have a dressing being capable of providing moist wound healing and at the same time being easy to remove in one piece without trauma for the patient and without sticking to the wound.

It is known that the healing of the wound can progress favorably only if the dressing does not adhere to the newly generated tissue and only if the exudates are removed while still leaving the wound moist.

2. Description of the Related Art

Such dressings may be in the form of a wound-contacting layer providing the desired properties. These dressings are commonly in the form of a textile sheet prepared from weak fibers, and consequently may have low mechanical strength and integrity. A well-known material used is polysaccharides such as carboxy methyl cellulose (CMC) or alginates. However, such fibers are weak fibers particularly when wetted and accordingly, a balance must be found between the conflicting desires for mechanical strength and integrity requiring high intensity needling and/or high basis density of the fibrous product.

The use of fibers often suffers from the drawback of a limited absorption and/or lack of cohesion, leading to difficulties with respect to removing the fibrous product from the wound as the product does not constitute a cohesive part.

Another well-known dressing is in the form of a greased fabric ensuring separation between the wound and an absorbent element. This eliminates the risk of the wound overgrowing the absorbent element rendering the removal of the dressing painful. This type of dressings may further comprise an amount of absorbent particles such as hydrocolloids. The presence of hydrocolloids renders the compress more hydrophilic which may be advantageous for the wound healing, especially when treating exuding wounds.

In U.S. Pat. No. 6,270,792 is disclosed a sterile non-stick compress comprising an open-mesh flexible fabric, the fabric being coated with a cohesive and non-stick gel. The gel is formed from a highly plasticized hydrophobic elastomer matrix having a dispersion of hydrophilic particles of a hydrocolloid.

From GB patent application No. 2 377 177 is disclosed a dressing comprising a thin layer of gel-forming fibers of alginate combined with a super-absorbent layer, such as a mixture of polyacrylate and viscose. The dressing is thick and is designed for heavily exuding wounds, due to the high absorbency.

GB patent application No. 2 221 620 discloses a dressing comprising synthetic fibers, the fibers being coated with an alginate solution. The solution has not been subjected to an ion exchange with e.g. Calcium, and will thus not form a coherent gel over the wound. Furthermore, fibers coated with an alginate solution will typically become stiff and brittle, and thus less flexible and soft against the skin/wound.

Thus there is still a need for a hydrophilic, lightweight wound dressing which is perfectly non-stick to regenerated tissue, which maintains optimum moisture conditions favorable to healing, whilst avoiding the risk of maceration and which is easy to remove in one piece.

### SUMMARY OF THE INVENTION

The invention relates to a wound dressing comprising a web of gel-forming fibers or fibers soluble in wound exudates, attached to a reinforcing layer.

The object of the invention is to provide a soft and flexible, easy handled, non-sticking and lightweight wound dressing for the use on low to high exudating wounds.

Another object of the invention is to provide an excellent release layer for donating active ingredients to a wound bed.

Yet another object of the invention is to provide a primary wound contacting layer, which does not adhere to the wound, and which is easy to remove in one piece.

### DETAILED DESCRIPTION OF THE PRESENT INVENTION

The invention relates to a wound dressing comprising a web of gel-forming fibers or fibers soluble in wound exudates, attached to a reinforcing layer wherein the density of the web is in the range of 5-60 g/m².

It has surprisingly been shown that the dressing of the invention is lightweight, skin-friendly and cohesive and provides good moist wound healing.

The lightweight properties of the dressing are achieved by supporting the fibrous web on a reinforcing layer. By the use of this layer it is possible to handle fibrous webs with a very low density without problems during manufacturing of the dressing.

The wound dressing of the invention comprising the web and the reinforcing layer has preferably a density lower than 100 g/m², more preferred lower than 80 g/m², even more preferred lower than 70 g/m² and most preferred lower than 50 g/m². In one embodiment of the invention the web has a density of 30 g/m² and the reinforcing layer 20 g/m², resulting in a density of the dressing of 50 g/m².

The web may be in the form of a three-dimensional structure such as an entangled web or the web may be a substantially two-dimensional structure such as a fibrous sheet or mat.

The dressing may be used by itself or it may be used in combination with or as a part of a further dressing. When treating highly exuding wound, it may be advantageous to combine the dressing of the invention with a secondary, highly absorbent dressing. The dressing of the invention may then serve as a wound-contacting layer, providing moist wound healing and transporting the exudates and slough to the secondary dressing. Thus maceration of the wound site may be avoided.

The dressing of the invention may serve as a wound contacting layer or a release layer for donating one or more active ingredients to a wound.

In one embodiment of the invention the dressing may be in the form of a cavity filler.

The dressing of the invention may be suitable for use on a broad range of wounds, from low to high exudating wounds. When used on high exudating wounds the dressing may preferably be combined with a further dressing comprising an absorbent layer.

The dressing of the invention may also be used on low exuding wounds. If the amount of exudates is too low to wet the dressing, the dressing may be prewetted before application to the wound. Prewetting of the dressing may also be used when the dressing is applied to intact skin, e.g. in the treatment of skin disorders such as psoriasis or callous skin, or when used for donation of an active ingredient.

The dressing of the invention may be suitable for acute wounds, burns and chronic wounds such as pressure sores, venous ulcers, leg ulcers or diabetic foot ulcers. Due to the high flexibility of the dressing of the invention it is very suitable for treatment of wounds on protruding body parts such as feet, fingers, toes, heels or elbows.

The dressing of the invention may be incorporated into another dressing, e.g. as a wound contacting layer. Such dressing may comprise a backing layer, an absorbent layer and the dressing of the invention as a wound-contacting layer. The dressing may further be provided with an adhesive layer for securing the dressing to the skin.

The fibers used in the dressing of the invention are gellable or soluble in wound exudate. Compared to commonly known alginate dressings the dressing of the invention comprises a lower amount of gellable or soluble fibers. But the fibers are combined with a reinforcing and strength giving material, which renders it possible to produce a lightweight dressing.

The dressing of the invention provides a skin-friendly surface and the manufacture of the dressing is cost-efficient compared to existing high-density alginate dressings. A high permeability is obtained by reducing the risk of gel blocking due to the low density of the dressing of the invention. Furthermore, the dressing is easy to remove in one piece, without leaving undesired residuals in the wound area.

The dressing of the invention is skin-friendly due to the no-stick properties and has very low risk of causing pressure sores, compared to other dressings.

A layer of gellable/soluble fibers with a density below 100 g/m² may not have the strength to be manufactured. Furthermore it may be difficult to handle when used on a wound and to be removed in one piece from the wound. Therefore a reinforcing layer may be added to the fibers. The presence of the reinforcing layer ensures the processability of the lightweight product and provides easy dressing change by removal in one piece.

The fibers are present in an amount providing a density of the web of 5 to 60 g/m², more preferred 5 to 50 g/m², even more preferred 15 to 40 g/m² and most preferred in the amount of 20 to 40 g/m².

In one embodiment of the invention the density of the web is 25-35 g/m², more preferred in the area of 30 g/m².

The fibers of the web may be selected from the group comprising of polysaccharide or polyacrylate fibers, preferably alginate, chitosane, modified chitosane, alginate fibers containing CMC or CMC fibers or derivatives or mixtures thereof.

The web may further comprise super absorbing particles or fibers.

The web may be in the form of a woven, non-woven, a knit, preferably non-woven.

In one embodiment of the invention the web is attached to the reinforcing layer by needling. Alternatively, the web may be attached by mechanical means or by thermal bonding or by the use of adhesive means. In another embodiment the attachment of the web to the reinforcing layer may be provided by a combination of the above-mentioned means.

By incorporating a reinforcing layer into the dressing a dressing with high cohesion is achieved in dry as well as wet condition. Furthermore, the typical use of cross folding of the fibers into a non-woven structure during the manufacture of the web in order to enhance the strength may be avoided.

The reinforcing layer may be any suitable layer providing the dressing with the desired properties with regard to strength and permeability. It may be in the form of a net, a foam, a film, a knit, a non-woven or woven material.

In a preferred embodiment of the invention the reinforcing layer is a non-woven net, preferably comprising polyethylene, Nylon, gauze, polyester, rubber, latex, cotton, textile or any other suitable fabric.

The reinforcing layer material is selected in such a manner, that the exudate and slough can permeate through the layer. In this way the exudates and slough may be transported away from the wound site and e.g. into an absorbent element of a secondary dressing.

The density of the reinforcing layer may be in the range of 5 to 200 g/m², preferably 10 to 100 g/m², more preferably 10 to 50 g/m², and most preferably 15 to 40 g/m² and very most preferred 20 to 30 g/m².

In one embodiment of the invention the dressing may comprise one or more active ingredients.

It is advantageous to provide a dressing of the invention with components for treatment or prophylaxis of formation of wounds and/or skin abnormalities, e.g. with emollients or an active constituent e.g. retinoids for treating or preventing formation of psoriasis, eczema, callous skin, corns, insect bites, acne or blisters. The dressing of the invention may also contain medicaments such as bacteriostatic or bactericide compounds, e.g. iodine, iodopovidone complexes, chloramine, chlorohexidine, silver salts, zinc or salts thereof, tissue-healing enhancing agents, e.g. RGD tripeptides and the like, enzymes for cleansing of wounds, e.g. proteases, pepsin, trypsin and the like, means for preventing the activity of proteolytic enzymes, pain relieving agents such as anaesthetica or analgetica, steroids, NSAIDS, such as Ibuprofen, Cox 2 inhibitors such as Celecoxib, odor reducing agents, such as charcoal, or agents having a cooling effect which is also considered an aspect of the invention.

The active ingredient may be incorporated into the dressing by means known in the art, such as coating, immobilizing, impregnating, chemical or mechanical bonding etc. The active ingredient may be incorporated into one or more of the components of the dressing, such as the reinforcing layer and/or the web.

The dressing may further comprise additives, rendering the incorporation of active ingredient easier, as well as providing sustained or controlled release of the active ingredient.

Especially in the treatment of chronic wounds, such as pressure sores and venous ulcers, it is often desired to incorporate antibacterial means in the dressing. These wounds may be infected, giving rise to delayed wound healing, odor problems and pain.

These antibacterial means, such as silver compounds, may be incorporated in the dressings, e.g. in the absorbent element or a wound contacting layer and may be released by more or less complicated release systems.

In a preferred embodiment the dressing comprises an antibacterial agent. Preferably the antibacterial agent is a source of silver, such as an Ag-complex or an Ag-salt.

In a particularly preferred embodiment the dressing comprises Ag—Ca-alginate or Ag—Na—CMC.

In a preferred embodiment of the invention the dressing comprises a pain-relieving agent.

The wound dressing may be produced by processing the gel forming/soluble fibers into at least one layer of carded fibers having a weight of maximum 50 g/m². The fiber layer may then be attached to the reinforcing material, e.g. by needling or bonding.

### EXAMPLE 1

Silver calcium alginate fibers (produced in accordance with GB Patent No. 2 370 226) with a content of silver of 10% w/w were carded to a web having a density of 15 g/m². The web was then placed on a polyethylene non-woven layer with a density of 30 g/m². The alginate layer was attached to the polyethylene layer by a conventional needling process to achieve a coherent web, which was cut into wound dressings.

The dressing showed antibacterial effect and was easily removed from the wound in one piece.

### EXAMPLE 2

A non-woven web of CMC fibers having a density of 40 g/m² was needled onto a 20 g/m² Polyethylene non-woven layer (providing reinforcement properties). The material was cut, packed and sterilized. The sample was used as a wound contact layer, thus preventing a secondary dressing from sticking to the wound. The wound dressing had an absorbency of 0.1 g/m².

### EXAMPLE 3

A non-woven carded web of Alginate fibers containing CMC having a density of 20 g/m² was needled onto a 40 g/m² polyethylene non-woven layer (giving reinforcement properties). The material was cut, packed and sterilized. The sample was used as a wound contact layer preventing the secondary dressing from sticking to the wound and at the same time providing moist wound healing properties.

### EXAMPLE 4

A non-woven carded web of standard calcium alginate fibers having a density of 30 g/m² was needled onto a 30 g/m² polyethylene non-woven layer (giving reinforcement properties). The material was cut, packed and sterilized. The sample was used as a wound contact layer, thus preventing a secondary dressing from sticking to the wound and at the same time providing a moist wound healing environment.

### EXAMPLE 5

A non-woven carded web of modified chitosan fibers (prepared as described in WO 01/24840) having a density of 30 g/m² was needled onto a 30 g/m² Nylon knitted layer (providing reinforcement properties). The material was cut, packed and sterilized. The sample was used as a wound contact layer, thus preventing a secondary dressing from sticking to the wound and at the same time providing a moist wound healing environment. The wound dressing had an absorbency of 0.2 g/m² in water.

### EXAMPLE 6

Silvered calcium alginate fibers as used in Example 1 (having a silver content of 25%) were mixed in a 1:1 ratio with standard calcium alginate fibers, obtaining a homogenous blend of fibers. A non-woven carded web of the above fibers having a density of 30 g/m² was needled onto a 30 g/m² polyethylene non-woven layer (providing reinforcement properties), obtaining a silver concentration of 0.4 mg/cm². The material was cut, packed and sterilized. The sample was used as a wound contact layer, thus providing a secondary dressing from sticking to the wound and at the same time providing a moist wound healing environment and antibacterial properties to control wound infection.

The antibacterial properties of the material were tested as described below. Isosensitest agar plates were inoculated with Ps.aeruginosa or St.aureus and incubated for 24 hr at 37° C. 10 mm circles of a sample of a dressing according to the invention were placed onto the agar plates. After 24 hours, the zone of inhibition was measured and the samples were moved to fresh inoculated and incubated agar plates. The zone of inhibition was measured after additional 24 hours (corresponding to a service time of 48 hours). This was repeated for additional 24 hours (corresponding to a service time of 72 hours).

The results are summarized in the below Table 1.

TABLE 1

| | Ps. Aeruginosa | | | St. Aureus | | |
|---|---|---|---|---|---|---|
| Tested material | 24 h | 48 h | 72 h | 24 h | 48 h | 72 h |
| Sample from Ex. 6 | 14 | 14 | 14 | 15 | 15 | 14 |

The dressing of the invention shows good antibacterial properties at all test hours.

### EXAMPLE 7

Silvered CMC fibers were prepared according to patent WO 03/022317A1. A non-woven carded web of the above fibers having a density of 40 g/m² was thermally bonded onto a 20 g/m² polyethylene non-woven layer (providing reinforcement properties, resulting in a silver concentration of 0.05 mg/cm². The material was cut, packed and sterilized. The sample was used as a wound contact layer, thus preventing a secondary dressing from sticking to the wound and at the same time providing a moist wound healing environment and antibacterial properties.

### EXAMPLE 8

A non-woven carded web of the silvered calcium alginate fibers described in Example 5 having a density of 50 g/m² was needled onto a 20 g/m² polyethylene non-woven layer (giving reinforcement properties), obtaining a silver concentration of 1.3 mg/cm². The material was cut, packed and sterilized. The sample was used as a wound contact layer, thus preventing a secondary dressing from sticking to the wound and at the same time providing antibacterial properties for treatment of wound infection.

### EXAMPLE 9

Standard calcium alginate fibers were suspended in ethanol containing Ibuprofen. The fibers was removed from the solution and dried. A non-woven carded web of the fibers having a density of 40 g/m² was needled onto a 20 g/m² polyethylene non-woven layer (giving reinforcement properties), obtaining an Ibuprofen concentration of 0.4 mg/cm². The material was cut, packed and sterilized. The sample was used as a wound contact layer, thus preventing a secondary dressing from sticking to the wound and at the same time providing a moist wound healing environment and pain relieving properties.

### EXAMPLE 10

The wound contact layer obtained in Example 6 was immersed in solution A (great excess) for 24 hours at 37 degrees. The sample was then removed from the solution by tweezers. It was observed that the product could easily be removed in one piece from solution A, with only shedding of some of the gelled/dissolved fibers.

1-10. (canceled)

11. A wound dressing comprising a web of gel-forming fibers or fibers soluble in wound exudates, attached to a reinforcing layer wherein the density of the web is in the range of 5-60 g/m².

12. A wound dressing according to claim 11 wherein the fibers are selected from the group consisting of polysaccharide or polyacrylate fibers, preferably alginate, chitosan, alginate containing CMC, or CMC fibers or derivatives or mixtures thereof.

13. A wound dressing according to claim 11 wherein the web is attached to the reinforcing layer by needling.

14. A wound dressing according to claim 11 wherein the web is attached to the reinforcing layer by thermal bonding.

15. A wound dressing according to claim 11 wherein the web is attached to the reinforcing layer by adhesive means.

16. A wound dressing according to claim 11 wherein the reinforcing layer is in the form of a net, a foam, a film, a non-woven or woven material.

17. A wound dressing according to claim 11 wherein the dressing comprises one or more active ingredients.

18. A wound dressing according to claim 17 wherein the active ingredient comprises an antibacterial agent.

19. A wound dressing according to claim 17 wherein the active ingredient comprises a pain-relieving agent.

20. A wound dressing according to claim 11 wherein the dressing comprises Ag—Ca-alginate and/or Ag—Na—CMC.
